(19) United States
(12) Patent Application Publication
(10) Pub. No.: US 2009/0221607 A1
(43) Pub. Date: Sep. 3, 2009

(54) Piperazine Derivatives

(75) Inventors: Connie L. Sun, Palo Alto, CA (US); Xiaoyuan Li, Los Altos, CA (US)

Correspondence Address: Paul N. King;King PLLC, 1201 Pennsylvania Ave. NW, Fifth Floor, Washington, DC, 20004, US

(73) Assignee: M's Science Corporation, Kobe (JP)

(21) Appl. No.: 12/223,444

(22) PCT Filed: Jan. 23, 2007
(86) PCT No.: PCT/US2007/002043

§ 371 Date: Jul. 31, 2008

Related U.S. Application Data
(60) Provisional application No. 60/773,391, filed on Feb. 15, 2006.

(51) Int. Cl. A61K 31/495; C07D 241/04

(52) U.S. Cl. 514/255.01; 544/398; 514/252.12; 544/391

(57) ABSTRACT

Compounds of general formula (I)

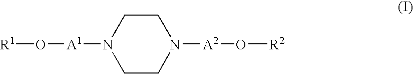

in which R¹, A¹, A² and R² have any of the meanings given in the specification, have affinity for sigma receptors and are useful in the treatment of disorders of the central nervous system.

The present invention relates to novel 1,4-piperazine derivatives, to processes for preparing the novel derivatives, to pharmaceutical compositions comprising the derivatives, and to the use of derivatives in the treatment of disorders of the central nervous system.

It has been disclosed in the scientific literature that certain disorders of the central nervous system may be treated using a modulator of sigma receptor function. Amongst compounds known to possess affinity for sigma ligands are certain piperazine derivatives.

WO 91/09594 discloses compounds having affinity for sigma receptors, certain of which are piperazine derivatives, and discloses that they are useful in the treatment of schizophrenia and other psychoses.

U.S. Pat. No. 5,736,546 discloses certain 1,4-(diphenylalkyl)-piperazines having one phenyl group unsubstituted and the other phenyl group substituted by two alkoxy groups. One of the compounds disclosed is 1-[2-(3,4-dimethoxyphenyl)ethyl]-4-(3-phenylpropyl)piperazine. It is also referred to in the scientific literature as SA 4503. The compounds of U.S. Pat. No. 5,736,546 are said to be useful in the treatment of dementia, depression, schizophrenia, anxiety neurosis, diseases accompanying abnormal immune response, cryptorrhea and digestive ulcer.

WO 2004/110387 discloses that sigma ligands, in particular SA 4503, are also useful in the treatment of patients to facilitate neuronal regeneration after onset of a neurodegenerative disease, such as ischemic stroke, traumatic brain injury or spinal chord injury.

U.S. Pat. No. 5,389,630 discloses certain diamine compounds having cerebral protective action. The compound of Example 50 is a piperazine derivative, but the vast majority of the exemplified compounds are homopiperazine derivatives. The mechanism of action of the compounds is not discussed.

French patent application publication number FR 2073277 generically discloses certain 1,4-disubstituted piperazine derivatives, which are said to possess a wide variety of pharmacological activities. One of the exemplified compounds is 1,4-bis-[2-(4-fluorophenoxy)ethyl]piperazine.

It has now been found that certain 1,4-piperazine derivatives, including the known compound 1,4-bis-[2-(4-fluorophenoxy)ethyl]piperazine, have high affinity for sigma receptors, in particular sigma-1 receptors.

According to one aspect, the present invention provides a compound of general formula (I)

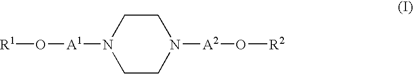

in which: —

R¹ represents a phenyl group that is substituted by one or two fluorine atoms;

A¹ represents (CH₂)ₘ or (CH₂)ₘ₋₁C(—O);

m is 2, 3, 4 or 5;

A² represents (CH₂)ₙ or C(═O)(CH₂)ₙ₋₁;

n is 2, 3, 4 or 5; and

R² represents a phenyl group that is unsubstituted or substituted by one, two or three substituents selected independently from (1-2C)alkylenedioxy, a halogen atom, a hydroxyl group, a cyano group, a (1-4C) alkyl group, a (3-6C)cycloalkyl group, a halo(1-4C) alkyl group, a (1-4C)alkoxy group, a (1-4C)alkoxy(1-4C)alkoxy group, a hydroxy(1-4C)alkoxy group, a (1-4C)alkylsulfanyl group, a (1-4C)alkylsulfonyl group, a halo(1-4C)alkoxy group and a halo(1-4C)alkylsulfonyl group;

or a pharmaceutically acceptable salt thereof,

provided that when A¹ and A² each represents (CH₂)₂, then R₁ and R₂ do not both represent 4-fluorophenyl.

Compounds according to the invention have been found to have high affinity for sigma receptors, in particular sigma-1 receptors.

As used herein, unless otherwise indicated, the term halogen atom includes fluorine, chlorine and bromine.

The term (1-2C)alkylenedioxy includes methylenedioxy and ethylenedioxy.

An example of a (1-4C) alkyl group is methyl.

The term halo(1-4C)alkyl as used herein includes perfluoro(1-4C)alkyl, such as trifluoromethyl.

An example of a (1-4C)alkoxy group is methoxy.

An example of a (1-4C)alkoxy(1-4C)alkoxy group is 2-methoxyethoxy.

An example of a hydroxy(1-4C)alkoxy group is hydroxymethyl.

The term halo(1-4C)alkoxy as used herein includes perfluoro(1-4C)alkoxy, such as trifluoromethoxy.

An example of a (1-4C)alkylsulfanyl group (also known as a (1-4C)alkylthio group) is methylsulfanyl.

An example of a (1-4C)alkylsulfonyl group is methylsulfonyl.

An example of a halo(1-4C)alkylsulfonyl group is trifluoromethylsulfonyl.

Referring to formula (I), examples of particular values for R¹ are 2-fluorophenyl, 3-fluorophenyl, 4-fluorophenyl and 2,4-difluorophenyl.

Examples of values for m are 2 and 3. An example of a particular value for m is 2.

Examples of particular values for n are 2 and 3.

In one embodiment, A¹ is (CH₂)ₘ, such as (CH₂)₂.

In another embodiment, A¹ is (CH₂)ₘ₋₁C(═O), such as (CH₂)C(═O) or (CH₂)₂C(═O). A² may represent, for example, (CH₂)ₙ.

In a further embodiment, R² represents a phenyl group that is substituted by one or two substituents selected independently from a fluorine atom, a cyano group, a fluoro(1-4C) alkyl group, a (1-4C)alkylsulfonyl group, a fluoro(1-4C)alkoxy group and a fluoro(1-4C)alkylsulfonyl group.

Examples of particular values for R² are 2-fluorophenyl, 4-fluorophenyl, 3-cyanophenyl, 4-cyanophenyl, 3-trifluoromethylphenyl, 4-trifluoromethylphenyl, 3-methylsulfonylphenyl, 4-methylsulfonylphenyl, 3-trifluoromethylsulfonylphenyl and 4-trifluoromethylsulfonylphenyl.

Particular mention is made of compounds of formula (I) in which R² represents 2-fluorophenyl or 4-fluorophenyl.

It will be appreciated that certain compounds of formula (I) contain a centre of asymmetry. These compounds may therefore exist and be isolated in the form of stereoisomers. The present invention provides a compound of formula (I) in any stereoisomeric form.

It will also be appreciated that the compounds of formula (I) or their pharmaceutically acceptable salts may be isolated in the form of a solvate, and accordingly that any such solvate is included within the scope of the present invention.

The compounds of general formula (I) can be prepared by conventional processes.

According to another aspect, therefore, the present invention provides a process for preparing a compound of general formula (I) or a pharmaceutically acceptable salt thereof, which comprises:

a) reacting a compound of general formula (II)

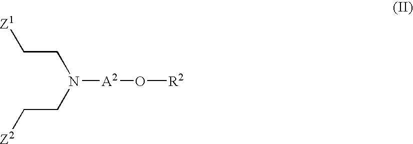

in which each of Z¹ and Z² independently represents a leaving atom or group, or a corresponding compound in which one or more substituents on R² are protected, with a compound of general formula (III)

R¹—O-A —NH₂ (III)

b) reacting a compound of general formula (IV)

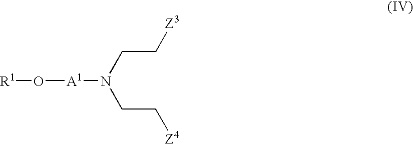

in which each of Z³ and Z⁴ independently represents a leaving atom or group, with a compound of general formula (V)

H₂N-A²-O—R² (V)

or a corresponding compound in which one or more substituents on R² are protected;

c) reacting a compound of general formula (VI)

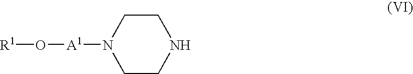

with a compound of general formula (VII)

Z⁵-A²-O—R² (VII)

in which Z⁵ represents a leaving atom or group;

d) reacting a compound of formula (VIII)

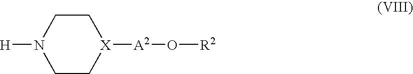

with a compound of general formula (IX)

R¹—O-A¹-Z⁶ (IX)

in which Z⁶ represents a leaving atom or group; or

e) for a compound of formula (I) in which A¹ represents (CH₂)ₘ and A² represents (CH₂)ₙ, reducing a corresponding compound of formula (I) in which A¹ represents (CH₂)ₘ₋₁C(═O) and/or A² represents C(═O)(CH₂)ₙ₋₁;

followed by removing any protecting group and, optionally, forming a pharmaceutically acceptable salt.

Referring to process step a), the leaving atoms or groups represented by Z¹ and Z² may be, for example, hydrocarbylsulfonyloxy groups, such as methanesulfonyloxy or p-toluenesulfonyloxy, or halogen atoms, such as chlorine atoms.

The reaction is conveniently performed at a temperature in the range of from 0 to 100° C., such as from 50 to 90° C. Convenient solvents include organic solvents, for example amides such as dimethylformamide. The reaction is conveniently performed in the presence of a base, for example an alkali metal carbonate such as potassium carbonate. The reaction may be performed in the presence of a catalyst, such as sodium iodide.

Compounds of general formula (III) can be prepared from the corresponding compounds of general formula (X)

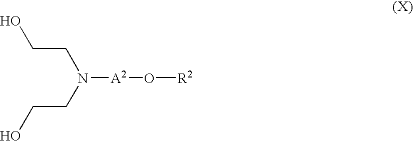

for example by reaction with thionyl chloride to afford a compound of formula (III) in which Z¹ and Z² represent chlorine atoms.

Compounds of general formula (X) can be prepared by reacting a compound of general formula (XI)

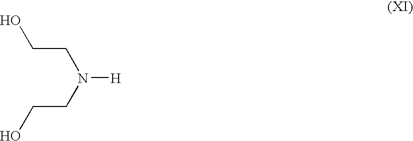

with a compound of general formula (VII), analogous to process c) described herein.

Process step b) may be performed by a method analogous to that described herein for process step a). The requisite starting materials may be obtained by reacting a compound of formula (XI) with a compound of formula (IX), then, for example, reacting the resultant diol with thionyl chloride to afford a compound of formula (IV).

Referring to process c), depending upon whether or not A² contains a carbonyl group, the leaving atom or group represented by Z⁵ may be, for example, a hydroxyl group, an acyloxy group or a halogen atom, such as a bromine atom. The reaction is conveniently performed at a temperature in the range of from 0 to 100° C. Convenient solvents include organic solvents, for example amides such as dimethylformamide. When the compound of formula (VII) is an alkyl halide, the reaction is conveniently performed in the presence of a base, for example an alkali metal carbonate such as caesium carbonate. When Z⁵ represents a hydroxyl group, the reaction is conveniently conducted under amide-bond coupling conditions, for example in the presence of 1-hydroxybenzotriazole and dicyclohexylcarbodiimide.

Compounds of formula (VI) may be prepared by deprotecting a compound of formula (XII)

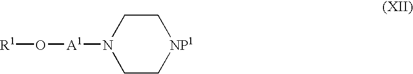

in which P¹ represents an amino protecting group, such as t-butoxycarbonyl. For example, a t-butoxycarbonyl group may be removed using trifluoroacetic acid. Alternatively, when the compound of formula (I) is symmetrical, it may be prepared in situ by reacting piperazine with two equivalents of a compound of formula (VII) and allowing the reaction to go to completion.

Process d) may be performed by the same method as process c).

Referring to process step e), the reducing agent can conveniently be a borane (BH₃), a borohydride reducing agent, such as sodium borohydride, or an alkali metal aluminium hydride, such as lithium aluminium hydride. The reduction is conveniently performed in the presence of a solvent such as an ether, for example tetrahydrofuran. The temperature at which the reduction is carried out is conveniently in the range of from −25 to 100° C., such as from −10 to 40° C.

A pharmaceutically acceptable salt may be formed by a conventional method, such as by reacting a compound of formula (I) with a pharmaceutically acceptable acid, such as hydrochloric acid.

Certain of the intermediates may be novel. The invention also provides all the novel intermediates disclosed herein.

The compounds of the invention may be administered by any convenient route, e.g. into the gastrointestinal tract (e.g. rectally or orally), the nose, lungs, musculature or vasculature or transdermally. The compounds may be administered in any convenient administrative form, e.g. tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the invention.

According to another aspect, the present invention provides a pharmaceutical composition, which comprises a compound of formula (I) or a pharmaceutically acceptable salt thereof, as defined hereinabove, together with a pharmaceutically acceptable diluent or carrier.

According to yet another aspect, the present invention provides a compound of formula (I), or a pharmaceutically acceptable salt thereof, as defined hereinabove, for use in therapy.

According to another aspect, the present invention provides the use of a compound of formula (I)

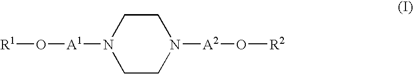

in which: —

R¹ represents a phenyl group that is substituted by one or two fluorine atoms;

A¹ represents (CH₂)ₘ or (CH₂)ₘ₋₁C(═O);

m is 2, 3, 4 or 5;

A² represents (CH₂)ₙ or C(═O)(CH₂)ₙ₋₁;

n is 2, 3, 4 or 5; and

R² represents a phenyl group that is unsubstituted or substituted by one, two or three substituents selected independently from (1-2C)alkylenedioxy, a halogen atom, a hydroxyl group, a cyano group, a (1-4C) alkyl group, a (3-6C)cycloalkyl group, a halo(1-4C) alkyl group, a (1-4C)alkoxy group, a (1-4C)alkoxy(1-4C)alkoxy group, a hydroxy(1-4C)alkoxy group, a (1-4C)alkylsulfanyl group, a (1-4C)alkylsulfonyl group, a halo(1-4C)alkoxy group and a halo(1-4C)alkylsulfonyl group;

or a pharmaceutically acceptable salt thereof, in the manufacture of a medicament for the treatment of a condition responsive to a modulator of sigma receptor function.

According to yet another aspect, the present invention provides a method of treating a condition responsive to a modulator of sigma receptor function in a patient requiring treatment, which comprises administering to said patient an effective amount of a compound of general formula (I)

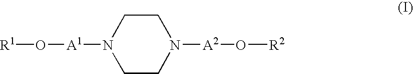

in which: —

R¹ represents a phenyl group that is substituted by one or two fluorine atoms;

A¹ represents (CH₂)ₘ or (CH₂)ₘ₋₁C(═O);

m is 2, 3, 4 or 5;

A² represents (CH₂)ₙ or C(═O)(CH₂)ₙ₋₁;

n is 2, 3, 4 or 5; and

R² represents a phenyl group that is unsubstituted or substituted by one, two or three substituents selected independently from (1-2C)alkylenedioxy, a halogen atom, a hydroxyl group, a cyano group, a (1-4C) alkyl group, a (3-6C)cycloalkyl group, a halo(1-4C) alkyl group, a (1-4C)alkoxy group, a (1-4C)alkoxy(1-4C)alkoxy group, a hydroxy(1-4C)alkoxy group, a (1-4C)alkylsulfanyl group, a (1-4C)alkylsulfonyl group, a halo(1-4C)alkoxy group and a halo(1-4C)alkylsulfonyl group;

or a pharmaceutically acceptable salt thereof.

The subject may be a human or a non-human animal, such as a non-human mammal, for example a cat, dog, horse, cow or sheep. Preferably the subject is a human.

The disorder responsive to a sigma receptor modulator may be, for example, a disorder of the central nervous system, such as a neurological disorder or a psychiatric disorder that has been linked to sigma receptors. Examples of neurological disorders include cerebral deficits subsequent to cardiac bypass surgery and grafting, cerebral ischemia (e.g. associated with stroke or cardiac arrest); spinal cord trauma; head trauma; multiple sclerosis, Alzheimers Disease; Huntington's Chorea; amyotrophic lateral sclerosis; AIDS-induced dementia; muscular spasms; convulsions; drug tolerance, withdrawal, and cessation (i.e. opiates, benzodiazepines, nicotine, cocaine, or ethanol); ocular damage and retinopathy; cognitive disorders; idiopathic and drug-induced Parkinson's Disease; pain; and movement disorders such as tardive dyskinesia. Examples of psychiatric disorders that are treated with a compound of formula I include schizophrenia, anxiety and related disorders (e.g. panic attack and stress-related disorders), depression, bipolar disorders, psychosis, and obsessive compulsive disorders.

The compounds according to the invention are of particular interest for use as neuroprotective agents and in the treatment of patients to facilitate neuronal regeneration and functional recovery after onset of a neurodegenerative disease, in particular ischemic stroke, traumatic brain injury, spinal chord injury and multiple sclerosis.

The dosage of the compounds of formula (I) will depend upon the nature and severity of the condition being treated, the administration route and the size and species of the subject. In general, quantities in the range of from 0.01 to 100 mg/kg bodyweight will be administered.

As used herein, the term “treatment” includes prophylactic use. The term “effective amount” refers to the amount of the compound of formula (I) that is effective to reduce or inhibit the development of the symptoms of the disorder being treated.

The compound according to the invention may be administered alone or in combination with another therapeutic agent having a different mode of action.

The ability of a compound to bind to a sigma receptor may be demonstrated by one or more of the following tests.

Sigma-1 (σ1) and sigma-2 (σ2) receptor binding assays are carried out in membranes from HEK-293 (Human Embryonic Kidney) cells.

### Membrane Preparation:

Confluent HEK-293 cells are harvested in PBS/5 mM EDTA. They are centrifuged at 2000 rpm for 5 min and then washed two times in PBS. Cells are homogenized in 20 mM Tris-HCL (pH=7.5) containing 5 mM EDTA, 0.5 mM PMSF and 0.5 μg/mL leupeptin using a Dounce homogenizer and sonicated for 5 minutes.

Nuclear debris and intact cells are removed by centrifugation at 3000 rpm for 10 minutes at 4° C. The supernatant is centrifuged at 12000 rpm for 30 minutes and the resulting pellet is resuspended in 25 mM Tris-HCL (pH=7.5), 25 mM Mg₂Cl, 10% sucrose containing 0.5 mM PMSF, 2 mM AEBSF, 1 mM EDTA, 130 μM bestatin, 14 μM E-64, 1 μM leupeptin and 0.3 mM aprotinin.

Proteins are determined using the Bio Rad Protein Assay Dye Reagent and the membranes are aliquoted and frozen at −80° C.

### σ1 Receptor Binding Assay

The binding assays are performed in 96-well plates.

σ1 receptors are labeled using the σ1 selective probe (+)-[³H] Pentazocine (Bowen W D et al, Mol Neuropharmacol 3, 117-126, 1993).

Total binding is determined by incubating 50 μg of HEK-293 cell membranes with 10 nM (+)-[³H]-pentazocine (Perkin-Elmer, 35 Ci/mmol) and assay buffer (50 mM Tris-HCl, pH=8.3) in a total volume of 200 μl. Non specific binding is determined in the presence of 10 μM unlabeled pentazocine. For competition experiments, 50 μl of displacing compound is added at 8 different concentrations. Incubations are carried out for 120 min at 37° C. Assays are terminated by dilution with ice-cold 10 mM Tris-HCl, pH=8.3 and vacuum filtration through glass fibers using a Skatron cell harvester from Molecular Devices. The filters are washed three times and the membrane-bound radioactivity is determined in a Microbeta scintillation counter.

Filters are soaked in 0.5% polyethyleneimine for 1 hour before use.

Specific binding is determined by subtraction of non specific binding from total binding. IC₅₀ values (concentration of competing ligand required for 50% inhibition of [³H]-pentazocine binding) are analyzed by non-linear regression fit using the GraphPad Prism software.

### σ2 Receptor Binding Assay

The binding assays are performed in 96-well plates

σ2 receptors are labeled using [³H] DTG (Di-o-tolylguanidine), under conditions in which σ1 receptors are masked with the σ1 selective compound pentazocine (Hellewell S B et al, Eur. J. Pharmacol, 268, 9-18, 1994).

Total binding is determined by incubating 50 μg of HEK-293 cell membranes with 10 nM [³H]-DTG (Perkin-Elmer, 58 Ci/mmol) in the presence of 10 μM pentazocine and assay buffer 50 mM Tris-HCl, pH=8.3) in a total volume of 200 μl. Non specific binding is determined in the presence of 10 μM unlabeled DTG. For competition experiments, 50 μl of displacing compound is added at 8 different concentrations. Incubations are carried out for 120 min at 37° C. Assays are terminated by dilution with ice-cold 10 mM Tris-HCl, pH=8.3 and vacuum filtration through glass fibers using a Skatron cell harvester from Molecular Devices. The filters are washed three times and the membrane-bound radioactivity is determined in a Microbeta scintillation counter.

Filters are soaked in 0.5% polyethyleneimine for 1 hour before use.

Specific binding is determined by subtraction of non specific binding from total binding. IC₅₀ values (concentration of competing ligand required for 50% inhibition of [³H]-DTG binding) are analyzed by non-linear regression fit using the GraphPad Prism software

The known compound 1,4-bis-[2-(4-fluorophenoxy)ethyl]piperazine and the compound of Example 1 have both been found to have an IC₅₀ of less than 700 nM in the σ1 receptor binding assay.

The following examples illustrate the invention.

### EXAMPLE 1

### 1,4-Bis-[2-(2-fluorophenoxy)ethyl]piperazine dihydrochloride

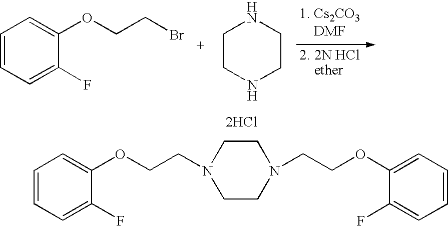

To piperazine (0.29 g, 3 mmol) in DMF (4 ml) was added Cs₂CO₃ (4.4 g, 13.5 mmol) and 1-(2-bromo-ethoxy)-2-fluoro-benzene (1.5 g, 6.8 mmol). The mixture was stirred at room temperature for a day, quenched with water, and extracted with dichloromethane. The organic layer was concentrated to give a white solid (0.88 g, 81%). Part of this solid (0.2 g) was suspended in ether and 2N HCl in ether (1 ml). The white solid was filtered to give the title compound (0.23 g, 96%).

¹H-NMR (400 MHz, CD₃OD-d₄): δ 3.70-3.76 (m, 12H), 4.49 (t, 4H), 7.03 (m, 2H), 7.18 (m, 6H).

The following compounds were prepared using the same method as descried in Example 1.

### EXAMPLE 2

### 1,4-Bis-[3-(4-fluorophenoxy)propyl]piperazine

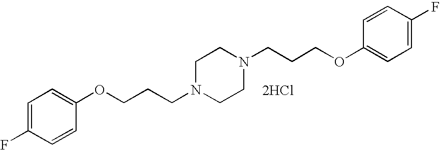

¹H-NMR (400 MHz, CD₃OD-d₄): δ 2.29 (m, 4H), 3.5-4.0 (m, 12H), 4.11 (t, 4H), 6.95 (m, 4H), 7.02 (m, 4H).

### EXAMPLE 3

### 1,4-Bis-[3-(2-fluorophenoxy)propyl]piperazine dihydrochloride

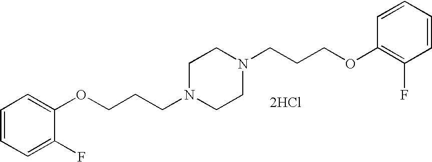

¹H-NMR (400 MHz, CD₃OD-d₄): δ 2.36 (m, 4H), 3.5-4.0 (m, 12H), 4.23 (t, 4H), 6.96 (m 2H), 7.12 (m, 6H).

### EXAMPLE 4

### 1-[2-(4-Fluorophenoxy)ethyl]-4-[3-(4-fluorophenoxy)propyl]piperazine dihydrochloride

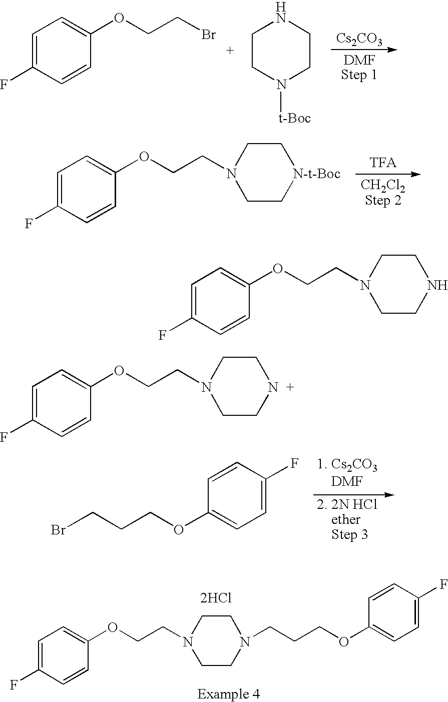

### Step 1: 4-[2-(4-Fluorophenoxy)ethyl]piperazine-1-carboxylic acid tert-butyl ester

To a solution of piperazine-1-carboxylic acid tert-butyl ester (1.86 g, 10 mmol) and 1-(2-bromoethoxy)-4-fluorobenzene (2.19 g, 10 mmol) in DMF (5 ml) was added Cs₂CO₃ (7.5 g, 20 mmol). The mixture was stirred at room temperature for a day, then quenched with water, and extracted with ethyl acetate. The organic layer was washed with water and brine, dried over anhydrous sodium sulphate, and concentrated to yield the title compound quantitatively as an oil (3.25 g) which was used without further purification.

¹H-NMR (400 MHz, DMSO-d₆): δ 1.39 (s, 9H), 2.42 (t, 4H), 2.69 (t, 2H), 3.31 (t, 4H), 4.04 (t, 2H), 6.95 (m, 2H), 7.10 (t, 2H).

### Step 2: 1-[2-(2-Fluorophenoxy)ethyl]piperazine

To the solution of 4-[2-(4-fluorophenoxy)ethyl]piperazine-1-carboxylic acid tert-butyl ester (3.2 g, 10 mmol) in dichloromethane (80 ml) was added with trifluoroacetic acid (15 ml). The reaction mixture was stirred at room temperature for 2 h and concentrated. The residue was treated with a saturated solution of NaHCO₃ extracted with dichloromethane. The organic layer was collected and concentrated to give the title compound quantitatively (5.0 g) as a white solid.

¹H-NMR (400 MHz, DMSO-d₆): δ 2.35 (m, 10H), 4.13 (t, 2H), 7.01 (t, 2H), 7.16 (t, 2H), 9.20 (b, 1H).

### Step 3: 1-[2-(4-Fluorophenoxy)ethyl]-4-[3-(4-fluorophenoxy)propyl]piperazine dihydrochloride

To 1-[2-(4-fluorophenoxy)ethyl]piperazine (0.148 mg, 0.5 mmol) in DMF (3 ml) was added Cs₂CO₃ (0.8 g, 2.5 mmol) and 1-(3-bromopropoxy)-4-fluorobenzene (139 mg, 0.6 mmol). The mixture was stirred at room temperature for a day, quenched with water and extracted with dichloromethane. The organic layer was concentrated and the residue was suspended in ether and 2N HCl in ether (2 ml). The white solid was filtered out to give the title compound (171 mg, 76%).

¹H-NMR (400 MHz, CD₃OD-d₄): δ 2.36 (m, 2H), 3.55 (t, 2H), 3.5-4.0 (b, 10H), 4.22 (t, 2H), 4.42 (t, 2H), 7.05 (m, 8H).

### EXAMPLE 5

### 2-(4-Fluorophenoxy)-1-{4-[2-(4-fluorophenoxy)acetyl]piperazin-1-yl}-ethanone

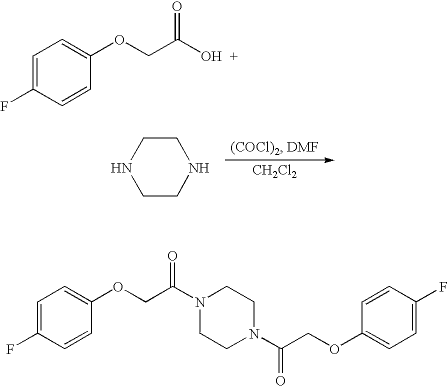

To the solution of (4-fluorophenoxy)acetic acid (8.5 g, 0.05 mol) in anhydrous dichloromethane (120 ml) was added oxalyl chloride (8.9 ml, 0.1 mol) and DMF (0.02 ml). The reaction mixture was stirred at room temperature for 1 h and concentrated. The residue was dissolved in anhydrous dichloromethane (60 ml). To this solution was added piperazine (1.9 g, 22 mmol) and triethylamine (11 ml, 0.08 mol). The reaction mixture was stirred overnight. The precipitate was filtered out, washed with methanol, and dried to afford the title compound quantitatively (9.16 g) as a white solid.

¹H-NMR 400 MHz, (DMSO-d₆): δ 5.48 (d, 8H), 4.85 (s, 4H), 6.95 (m, 4H), 7.11 (m, 4H)

The following compounds and their pharmaceutically acceptable salts can be prepared using the same method as descried in Example 1.
4-(2-{4-[2-(4-Fluorophenoxy)ethyl]-piperazin-1-yl}ethoxy)benzonitrile
4-(2-{4-[2-(2-Fluorophenoxy)ethyl]piperazin-1-yl}ethoxy)benzonitrile
4-(2-{4-[2-(2,4-Difluorophenoxy)ethyl]piperazin-1-yl}ethoxy)benzonitrile
3-(2-{4-[2-(4-Fluorophenoxy)ethyl]piperazin-1-yl}ethoxy)benzonitrile
3-(2-{4-[2-(2-Fluorophenoxy)ethyl]piperazin-1-yl}ethoxy)benzonitrile
3-(2-{4-[2-(2,4-Difluorophenoxy)ethyl]piperazin-1-yl}ethoxy)benzonitrile
1-[2-(4-Fluorophenoxy)ethyl]-4-[2-(3-trifluoromethylphenoxy)ethyl]piperazine
1-[2-(2-Fluorophenoxy)ethyl]-4-[2-(3-trifluoromethylphenoxy)ethyl]piperazine
1-[2-(2,4-Difluorophenoxy)ethyl]-4-[2-(3-trifluoromethylphenoxy)ethyl]piperazine
1-[2-(4-Fluorophenoxy)ethyl]-4-[2-(4-trifluoromethylphenoxy)ethyl]piperazine
1-[2-(2-Fluorophenoxy)ethyl]-4-[2-(4-trifluoromethylphenoxy)ethyl]piperazine
1-[2-(2,4-Difluorophenoxy)ethyl]-4-[2-(4-trifluoromethylphenoxy)ethyl]piperazine
1-[2-(4-Fluorophenoxy)ethyl]-4-[2-(4-methanesulfonylphenoxy)ethyl]piperazine
1-[2-(2-Fluorophenoxy)ethyl]-4-[2-(4-methanesulfonylphenoxy)ethyl]piperazine
1-[2-(2,4-Difluorophenoxy)ethyl]-4-[2-(4-methanesulfonylphenoxy)ethyl]piperazine
1-[2-(4-Fluorophenoxy)ethyl]-4-[2-(3-methanesulfonylphenoxy)ethyl]piperazine
1-[2-(2-Fluorophenoxy)ethyl]-4-[2-(3-methanesulfonylphenoxy)ethyl]piperazine
1-[2-(2,4-Difluorophenoxy)ethyl]-4-[2-(3-methanesulfonylphenoxy)ethyl]piperazine
1-[2-(4-Fluorophenoxy)ethyl]-4-[2-(4-trifluoromethanesulfonylphenoxy)ethyl]piperazine
1-[2-(2-Fluorophenoxy)ethyl]-4-[2-(4-trifluoromethanesulfonylphenoxy)ethyl]piperazine
1-[2-(2,4-Difluorophenoxy)ethyl]-4-[2-(4-trifluoromethanesulfonylphenoxy)ethyl]piperazine
1-[2-(4-Fluorophenoxy)ethyl]-4-[2-(3-trifluoromethanesulfonylphenoxy)ethyl]piperazine
1-[2-(2-Fluorophenoxy)ethyl]-4-[2-(3-trifluoromethanesulfonylphenoxy)ethyl]piperazine
1-[2-(2,4-Difluorophenoxy)ethyl]-4-[2-(3-trifluoromethanesulfonylphenoxy)ethyl]piperazine
4-(3-{4-[2-(4-Fluorophenoxy)ethyl]piperazin-1-yl}propoxy)benzonitrile
4-(3-{4-[2-(2-Fluorophenoxy)ethyl]piperazin-1-yl}propoxy)benzonitrile
4-(3-{4-[2-(2,4-Difluorophenoxy)ethyl]piperazin-1-yl}propoxy)benzonitrile
3-(3-{4-[2-(4-Fluorophenoxy)ethyl]piperazin-1-yl}propoxy)benzonitrile
3-(3-{4-[2-(2-Fluorophenoxy)ethyl]piperazin-1-yl}propoxy)benzonitrile
3-(3-{4-[2-(2,4-Difluorophenoxy)ethyl]piperazin-1-yl}propoxy)benzonitrile
1-[2-(4-Fluorophenoxy)ethyl]-4-[3-(4-trifluoromethylphenoxy)propyl]piperazine
1-[2-(2-Fluorophenoxy)ethyl]-4-[3-(4-trifluoromethylphenoxy)propyl]piperazine
1-[2-(2,4-Difluorophenoxy)ethyl]-4-[3-(4-trifluoromethylphenoxy)propyl]piperazine
1-[2-(4-Fluorophenoxy)ethyl]-4-[3-(3-trifluoromethylphenoxy)propyl]piperazine
1-[2-(2-Fluorophenoxy)ethyl]-4-[3-(3-trifluoromethylphenoxy)propyl]piperazine
1-[2-(2,4-Difluorophenoxy)-ethyl]-4-[3-(3-trifluoromethylphenoxy)propyl]piperazine
1′-[2-(4-Fluorophenoxy)ethyl]-4-[3-(4-methanesulfonylphenoxy)propyl]piperazine
1-[2-(2-Fluorophenoxy)ethyl]-4-[3-(4-methanesulfonylphenoxy)propyl]piperazine
1-[2-(2,4-Difluorophenoxy)ethyl]-4-[3-(4-methanesulfonylphenoxy)propyl]piperazine
1-[2-(4-Fluorophenoxy)ethyl]-4-[3-(3-methanesulfonylphenoxy)propyl]piperazine
1-[2-(2,4-Fluorophenoxy)ethyl]-4-[3-(3-methanesulfonylphenoxy)propyl]piperazine
1-[2-(2,4-Difluorophenoxy)ethyl]-4-[3-(3-methanesulfonylphenoxy)propyl]piperazine
1-[2-(4-Fluorophenoxy)ethyl]-4-[3-(4-trifluoromethanesulfonylphenoxy)propyl]piperazine
1-[2-(2-Fluorophenoxy)ethyl]-4-[3-(4-trifluoromethanesulfonylphenoxy)propyl]piperazine
1-[2-(2,4-Difluorophenoxy)ethyl]-4-[3-(4-trifluoromethanesulfonylphenoxy)propyl]-piperazine,
1-[2-(4-Fluorophenoxy)ethyl]-4-[3-(3-trifluoromethanesulfonylphenoxy)propyl]piperazine
1-[2-(2-Fluorophenoxy)ethyl]-4-[3-(3-trifluoromethanesulfonylphenoxy)propyl]piperazine; and
1-[2-(2,4-Difluorophenoxy)ethyl]-4-[3-(3-trifluoromethanesulfonylphenoxy)propyl]-piperazine.

1-15. (canceled)

16. A compound of general formula (I) 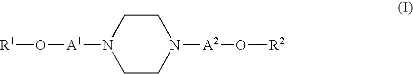
in which: —
R¹ represents a phenyl group that is substituted by one or two fluorine atoms;
A¹ represents (CH₂)ₘ or (CH₂)ₘ₋₁C(═O);
m is 2, 3, 4 or 5;
A² represents (CH₂)ₙ or C(═O)(CH₂)ₙ₋₁;
n is 2, 3, 4 or 5; and
R² represents a phenyl group that is unsubstituted or substituted by one, two or three substituents selected independently from (1-2C)alkylenedioxy, a halogen atom, a hydroxyl group, a cyano group, a (1-4C) alkyl group, a (3-6C)cycloalkyl group, a halo(1-4C) alkyl group, a (1-4C)alkoxy group, a (1-4C)alkoxy(1-4C)alkoxy group, a hydroxy(1-4C)alkoxy group, a (1-4C)alkylsulfanyl group, a (1-4C)alkylsulfonyl group, a halo(1-4C)alkoxy group, and a halo(1-4C)alkylsulfonyl group;
or a pharmaceutically acceptable salt thereof,
or a stereoisomeric form or a polymorphic form of said compound or of said salt;
provided that when A¹ and A² each represents (CH₂)₂, then R₁ and R₂ do not both represent 4-fluorophenyl.

17. A compound as claimed in claim 16, in R¹ represents a 2-fluorophenyl, 3-fluorophenyl, 4-fluorophenyl, or 2,4-difluorophenyl group.

18. A compound as claimed in claim 16 or claim 17 in which m is 2.

19. A compound as claimed in claim 16 or claim 17 in which m is 3.

20. A compound as claimed in any one of claims 16 and 17 in which A¹ represents (CH₂)ₘ.

21. A compound as claimed in any one of claims 16 and 17 in which n is 2.

22. A compound as claimed in any one of claims 16 and 17 in which n is 3.

23. A compound as claimed in any one of claims 16 and 17 in which A represents (CH₂)ₙ.

24. A compound of general formula (I) 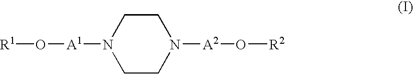
in which: —
R¹ represents a phenyl group that is substituted by one or two fluorine atoms;
A¹ represents (CH₂)ₘ or (CH₂)ₘ₋₁C(═O);
m is 2, 3, 4 or 5;
A represents (CH₂)ₙ or C(═O)(CH₂)ₙ₋₁;
n is 2, 3, 4 or 5; and
R² represents a phenyl group that is substituted by one or two substituents selected independently from a fluorine atom, a cyano group, a fluoro(1-4C) alkyl group, a (1-4C)alkylsulfonyl group, a fluoro(1-4C)alkoxy group and a fluoro(1-4C)alkylsulfonyl group;
or a pharmaceutically acceptable salt thereof,
or a stereoisomeric form or a polymorphic form of said compound or of said salt;
provided that when A¹ and A² each represents (CH₂)₂, then R₁ and R₂ do not both represent 4-fluorophenyl.

25. A compound as claimed in claim 17, in which R² represents a substituent selected from the group consisting of 2-fluorophenyl, 4-fluorophenyl, 3-cyanophenyl, 4-cyanophenyl, 3-trifluoromethylphenyl, 4-trifluoromethylphenyl, 3-methylsulfonylphenyl, 4-methylsulfonylphenyl, 3-trifluoromethylsulfonylphenyl, and 4-trifluoromethylsulfonylphenyl.

26. A compound as claimed in claim 25 in which R² represents 2-fluorophenyl.

27. A compound as claimed in claim 25 in which R² represents 4-fluorophenyl.

28. A compound selected from the group consisting of:
1,4-Bis-[2-(2-fluorophenoxy)ethyl]piperazine dihydrochloride,
1,4-Bis-[3-(4-fluorophenoxy)propyl]piperazine,
1,4-Bis-[3-(2-fluorophenoxy)propyl]piperazine dihydrochloride,
1-[2-(4-Fluorophenoxy)ethyl]-4-[3-(4-fluorophenoxy)propyl]piperazine dihydrochloride,
and 2-(4-Fluorophenoxy)-1-{4-[2-(4-fluorophenoxy)acetyl]piperazin-1-yl}-ethanone,
or a pharmaceutically acceptable salt of the selected compound, including a stereoisomeric form or a polymorphic form of said compound or of said salt.

29. A compound selected from the group consisting of:
4-(2-{4-[2-(4-Fluorophenoxy)ethyl]piperazin-1-yl}ethoxy)benzonitrile,
4-(2-{4-[2-(2-Fluorophenoxy)ethyl]piperazin-1-yl}ethoxy)benzonitrile,
4-(2-{4-[2-(2,4-Difluorophenoxy)ethyl]piperazin-1-yl}ethoxy)benzonitrile,
3-(2-{4-[2-(4-Fluorophenoxy)ethyl]piperazin-1-yl}ethoxy)benzonitrile,
3-(2-{4-[2-(2-Fluorophenoxy)ethyl]piperazin-1-yl}ethoxy)benzonitrile,
3-(2-{4-[2-(2,4-Difluorophenoxy)ethyl]piperazin-1-yl}ethoxy)benzonitrile,
1-[2-(4-Fluorophenoxy)ethyl]-4-[2-(3-trifluoromethylphenoxy)ethyl]piperazine,
1-[2-(2-Fluorophenoxy)ethyl]-4-[2-(3-trifluoromethylphenoxy)ethyl]piperazine,
1-[2-(2,4-Difluorophenoxy)ethyl]-4-[2-(3-trifluoromethylphenoxy)ethyl]piperazine,
1-[2-(4-Fluorophenoxy)ethyl]-4-[2-(4-trifluoromethylphenoxy)ethyl]piperazine,
1-[2-(2-Fluorophenoxy)ethyl]-4-[2-(4-trifluoromethylphenoxy)ethyl]piperazine,
1-[2-(2,4-Difluorophenoxy)ethyl]-4-[2-(4-trifluoromethylphenoxy)ethyl]piperazine,
1-[2-(4-Fluorophenoxy)ethyl]-4-[2-(4-methanesulfonylphenoxy)ethyl]piperazine,
1-[2-(2-Fluorophenoxy)ethyl]-4-[2-(4-methanesulfonylphenoxy)ethyl]piperazine,
1-[2-(2,4-Difluorophenoxy)ethyl]-4-[2-(4-methanesulfonylphenoxy)ethyl]piperazine,
1-[2-(4-Fluorophenoxy)ethyl]-4-[2-(3-methanesulfonylphenoxy)ethyl]piperazine,
1-[2-(2-Fluorophenoxy)ethyl]-4-[2-(3-methanesulfonylphenoxy)ethyl]piperazine,
1-[2-(2,4-Difluorophenoxy)ethyl]-4-[2-(3-methanesulfonylphenoxy)ethyl]piperazine,
1-[2-(4-Fluorophenoxy)ethyl]-4-[2-(4-trifluoromethanesulfonylphenoxy)ethyl]piperazine,
1-[2-(2-Fluorophenoxy)ethyl]-4-[2-(4-trifluoromethanesulfonylphenoxy)ethyl]piperazine,
1-[2-(2,4-Difluorophenoxy)ethyl]-4-[2-(4-trifluoromethanesulfonylphenoxy)ethyl]piperazine,
1-[2-(4-Fluorophenoxy)ethyl]-4-[2-(3-trifluoromethanesulfonylphenoxy)ethyl]piperazine,
1-[2-(2-Fluorophenoxy)ethyl]-4-[2-(3-trifluoromethanesulfonylphenoxy)ethyl]piperazine,
1-[2-(2,4-Difluorophenoxy)ethyl]-4-[2-(3-trifluoromethanesulfonylphenoxy)ethyl]piperazine,
4-(3-{4-[2-(4-Fluorophenoxy)ethyl]piperazin-1-yl}propoxy)benzonitrile,
4-(3-{4-[2-(2-Fluorophenoxy)ethyl]piperazin-1-yl}propoxy)benzonitrile,
4-(3-{4-[2-(2,4-Difluorophenoxy)ethyl]piperazin-1-yl}propoxy)benzonitrile,
3-(3-{4-[2-(4-Fluorophenoxy)ethyl]piperazin-1-yl}propoxy)benzonitrile,
3-(3-{4-[2-(2-Fluorophenoxy)ethyl]piperazin-1-yl}propoxy)benzonitrile,
3-(3-{4-[2-(2,4-Difluorophenoxy)ethyl]piperazin-1-yl}propoxy)benzonitrile,
1-[2-(4-Fluorophenoxy)ethyl]-4-[3-(4-trifluoromethylphenoxy)propyl]piperazine,
1-[2-(2-Fluorophenoxy)ethyl]-4-[3-(4-trifluoromethylphenoxy)propyl]piperazine,
1-[2-(2,4-Difluorophenoxy)ethyl]-4-[3-(4-trifluoromethylphenoxy)propyl]piperazine,
1-[2-(4-Fluorophenoxy)ethyl]-4-[3-(3-trifluoromethylphenoxy)propyl]piperazine,
1-[2-(2-Fluorophenoxy)ethyl]-4-[3-(3-trifluoromethylphenoxy)propyl]piperazine,
1-[2-(2,4-Difluorophenoxy)ethyl]-4-[3-(3-trifluoromethylphenoxy)propyl]piperazine,
1-[2-(4-Fluorophenoxy)ethyl]-4-[3-(4-methanesulfonylphenoxy)propyl]piperazine,
1-[2-(2-Difluorophenoxy)ethyl]-4-[3-(4-methanesulfonylphenoxy)propyl]piperazine,
1-[2-(2,4-Difluorophenoxy)ethyl]-4-[3-(4-methanesulfonylphenoxy)propyl]piperazine,
1-[2-(4-Fluorophenoxy)ethyl]-4-[3-(3-methanesulfonylphenoxy)propyl]piperazine,
1-[2-(2-Fluorophenoxy)ethyl]-4-[3-(3-methanesulfonylphenoxy)propyl]piperazine,
1-[2-(2,4-Difluorophenoxy)ethyl]-4-[3-(3-trifluoromethanesulfonylphenoxy)propyl]piperazine,
1-[2-(4-Fluorophenoxy)ethyl]-4-[3-(4-trifluoromethanesulfonylphenoxy)propyl]piperazine,
1-[2-(2-Fluorophenoxy)ethyl]-4-[3-(4-trifluoromethanesulfonylphenoxy)propyl]piperazine,
1-[2-(2,4-Difluorophenoxy)ethyl]-4-[3-(4-trifluoromethanesulfonylphenoxy)propyl]piperazine,
1-[2-(4-Fluorophenoxy)ethyl]-4-[3-(3-trifluoromethanesulfonylphenoxy)propyl]piperazine,
1-[2-(2-Fluorophenoxy)ethyl]-4-[3-(3-trifluoromethanesulfonylphenoxy)propyl]piperazine, and
1-[2-(2,4-Difluorophenoxy)ethyl]-4-[3-(3-trifluoromethanesulfonylphenoxy)propyl]piperazine,
or a pharmaceutically acceptable salt of the selected compound, or a stereoisomeric form or a polymorphic form of said compound or of said salt.

30. A process for preparing a compound of the general formula (I) 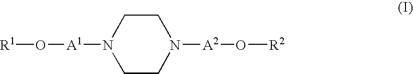
in which: —
R¹ represents a phenyl group that is substituted by one or two fluorine atoms;
A¹ represents (CH₂)ₘ or (CH₂)ₘ₋₁C(═O);
m is 2, 3, 4 or 5;
A represents (CH₂)ₙ or C(═O)(CH₂)ₙ₋₁;
n is 2, 3, 4 or 5; and
R¹ represents a phenyl group that is unsubstituted or substituted by one, two or three substituents selected independently from (1-2C)alkylenedioxy, a halogen atom, a hydroxyl group, a cyano group, a (1-4C) alkyl group, a (3-6C)cycloalkyl group, a halo(1-4C) alkyl group, a (1-4C)alkoxy group, a (1-4C)alkoxy(1-4C)alkoxy group, a hydroxy(1-4C)alkoxy group, a (1-4C)alkylsulfanyl group, a (1-4C)alkylsulfonyl group, a halo(1-4C)alkoxy group, and a halo(1-4C)alkylsulfonyl group;
or a pharmaceutically acceptable salt thereof,
or a stereoisomeric form or a polymorphic form of said compound or of said salt;
provided that when A¹ and A² each represents (CH₂)₂, then R₁ and R₂ do not both represent 4-fluorophenyl,
comprising a process selected from the group consisting of:
a) reacting a compound of general formula (II) 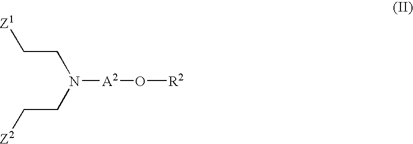
in which each of Z¹ and Z² independently represents a leaving atom or group, or a corresponding compound in which one or more substituents on R² are protected, with a compound of general formula (III)
R¹—O-A¹-NH₂ (III);
b) reacting a compound of general formula (IV) 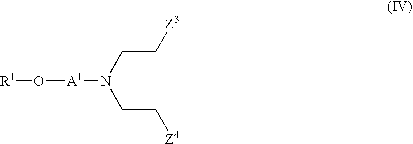
in which each of Z³ and Z⁴ independently represents a leaving atom or group, with a compound of general formula (V)
H₂N-A²-O—R² (V)
or a corresponding compound in which one or more substituents on R² are protected;
c) reacting a compound of general formula (VI) 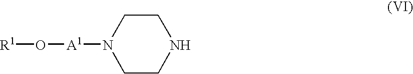
with a compound of general formula (VII)
Z⁵-A²-O—R² VII)
in which Z⁵ represents a leaving atom or group;
d) reacting a compound of formula (VIII) 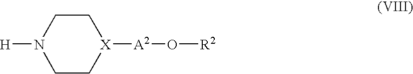
with a compound of general formula (IX)
R¹—O-A¹-Z⁶ (IX)
in which Z⁶ represents a leaving atom or group; and
e) for a compound of formula (I) in which A¹ represents (CH₂)ₘ and A² represents (CH₂)ₙ, reducing a corresponding compound of formula (I) in which A¹ represents (CH₂)ₘ₋₁C(═O) and/or A² represents C(═O)(CH₂)ₙ₋₁;
the selected process followed by removing any protecting group, and, optionally, forming a pharmaceutically acceptable salt.

31. A pharmaceutical composition, which comprises a compound as claimed in any one of claims 16, 17, 24, 28, or 29 and a pharmaceutically acceptable diluent or carrier.

32. A method of treating a condition responsive to a modulator of sigma receptor function in a patient requiring treatment, which comprises administering to said patient an effective amount of a compound of general formula (I) 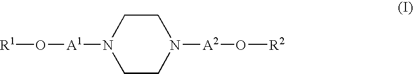
in which: —
R¹ represents a phenyl group that is substituted by one or two fluorine atoms;
A¹ represents (CH₂)ₘ or (CH₂)ₘ₋₁C(═O);
m is 2, 3, 4 or 5;
A represents (CH₂)ₙ or C(═O)(CH₂)ₙ₋₁;
n is 2, 3, 4 or 5; and
R² represents a phenyl group that is unsubstituted or substituted by one, two or three substituents selected independently from (1-2C)alkylenedioxy, a halogen atom, a hydroxyl group, a cyano group, a (1-4C) alkyl group, a (3-6C)cycloalkyl group, a halo(1-4C) alkyl group, a (1-4C)alkoxy group, a (1-4C)alkoxy(1-4C)alkoxy group, a hydroxy(1-4C)alkoxy group, a (1-4C)alkylsulfanyl group, a (1-4C)alkylsulfonyl group, a halo(1-4C)alkoxy group and a halo(1-4C)alkylsulfonyl group;
or a pharmaceutically acceptable salt thereof, or a stereoisomeric form or a polymorphic form of said compound or of said salt,
provided that when A¹ and A² each represents (CH₂)₂, then R₁ and R₂ do not both represent 4-fluorophenyl.

33. The method according to claim 32 which comprises administering to said patient an effective amount of a compound of the general formula (I) in which R¹ represents a 2-fluorophenyl, 3-fluorophenyl, 4-fluorophenyl, or 2,4-difluorophenyl group.

34. A method of treating a condition as in any one of claims 32 and 33, wherein m is 2 in the compound of general formula (I).

35. A method of treating a condition as in any one of claims 32 and 33, wherein m is 3 in the compound of general formula (I).

36. A method of treating a condition as in any one of claims 32 and 33, wherein A¹ represents (CH₂)ₘ in the compound of general formula (I).

37. A method of treating a condition as in any one of claims 32 and 33, wherein n is 2 in the compound of general formula (I).

38. A method of treating a condition as in any one of claims 32 and 33, wherein n is 3 in the compound of general formula (I).

39. A method of treating a condition as in any one of claims 32 and 33, wherein A² represents (CH₂)ₙ in the compound of general formula (I).

40. A method of treating a condition responsive to a modulator of sigma receptor function in a patient requiring treatment, which comprises administering to said patient an effective amount of a compound of general formula (I) 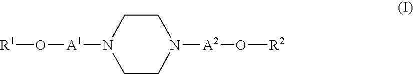
in which: —
R¹ represents a phenyl group that is substituted by one or two fluorine atoms;
A¹ represents (CH₂)ₘ or (CH₂)ₘ₋₁C(═O);
m is 2, 3, 4 or 5;
A represents (CH₂)ₙ or C(═O)(CH₂)ₙ₋₁;
n is 2, 3, 4 or 5; and
R² represents a phenyl group that is substituted by one or two substituents selected independently from a fluorine atom, a cyano group, a fluoro(1-4C) alkyl group, a (1-4C)alkylsulfonyl group, a fluoro(1-4C)alkoxy group and a fluoro(1-4C)alkylsulfonyl group;
or a pharmaceutically acceptable salt thereof,
or a stereoisomeric form or a polymorphic form of said compound or of said salt;
provided that when A¹ and A² each represents (CH₂)₂, then R₁ and R₂ do not both represent 4-fluorophenyl.

41. A method of treating a condition as claimed in claim 40, in which R² of the compound represents a substituent selected from the group of 2-fluorophenyl, 4-fluorophenyl, 3-cyanophenyl, 4-cyanophenyl, 3-trifluoromethylphenyl, 4-trifluoromethylphenyl, 3-methylsulfonylphenyl, 4-methylsulfonylphenyl, 3-trifluoromethylsulfonylphenyl, and 4-trifluoromethylsulfonylphenyl.

42. A compound as claimed in claim 41, in which R² of the compound represents 2-fluorophenyl.

43. A compound as claimed in claim 41, in which R² of the compound represents 4-fluorophenyl.

44. A method of treating a condition responsive to a modulator of sigma receptor function in a patient requiring treatment, which comprises administering to said patient an effective amount of a compound selected from the group consisting of:
1,4-Bis-[2-(2-fluorophenoxy)ethyl]piperazine dihydrochloride,
1,4-Bis-[3-(4-fluorophenoxy)propyl]piperazine,
1,4-Bis-[3-(2-fluorophenoxy)propyl]piperazine dihydrochloride,
1-[2-(4-Fluorophenoxy)ethyl]-4-[3-(4-fluorophenoxy)propyl]piperazine dihydrochloride,
and 2-(4-Fluorophenoxy)-1-{4-[2-(4-fluorophenoxy)acetyl]piperazin-1-yl}-ethanone,
or a pharmaceutically acceptable salt of the selected compound,
or a stereoisomeric form or a polymorphic form of said compound or of said salt.

45. A method of treating a condition responsive to a modulator of sigma receptor function in a patient requiring treatment, which comprises administering to said patient an effective amount of a compound selected from the group consisting of:
4-(2-{4-[2-(4-Fluorophenoxy)ethyl]-piperazin-1-yl}ethoxy)benzonitrile,
4-(2-{4-[2-(2-Fluorophenoxy)ethyl]piperazin-1-yl}ethoxy)benzonitrile,
4-(2-{4-[2-(2,4-Difluorophenoxy)ethyl]piperazin-1-yl}ethoxy)benzonitrile,
3-(2-{4-[2-(4-Fluorophenoxy)ethyl]piperazin-1-yl}ethoxy)benzonitrile,
3-(2-{4-[2-(2-Fluorophenoxy)ethyl]piperazin-1-yl}ethoxy)benzonitrile,
3-(2-{4-[2-(2,4-Difluorophenoxy)ethyl]piperazin-1-yl}ethoxy)benzonitrile,
1-[2-(4-Fluorophenoxy)ethyl]-4-[2-(3-trifluoromethylphenoxy)ethyl]piperazine,
1-[2-(2-Fluorophenoxy)ethyl]-4-[2-(3-trifluoromethylphenoxy)ethyl]piperazine,
1-[2-(2,4-Difluorophenoxy)ethyl]-4-[2-(3-trifluoromethylphenoxy)ethyl]piperazine,
1-[2-(4-Fluorophenoxy)ethyl]-4-[2-(4-trifluoromethylphenoxy)ethyl]piperazine,
1-[2-(2-Fluorophenoxy)ethyl]-4-[2-(4-trifluoromethylphenoxy)ethyl]piperazine,
1-[2-(2,4-Difluorophenoxy)ethyl]-4-[2-(4-trifluoromethylphenoxy)ethyl]piperazine,
1-[2-(4-Fluorophenoxy)ethyl]-4-[2-(4-methanesulfonylphenoxy)ethyl]piperazine,
1-[2-(2-Fluorophenoxy)ethyl]-4-[2-(4-methanesulfonylphenoxy)ethyl]piperazine,
1-[2-(2,4-Difluorophenoxy)ethyl]-4-[2-(4-methanesulfonylphenoxy)ethyl]piperazine,
1-[2-(4-Fluorophenoxy)ethyl]-4-[2-(3-methanesulfonylphenoxy)ethyl]piperazine,
1-[2-(2-Fluorophenoxy)ethyl]-4-[2-(3-methanesulfonylphenoxy)ethyl]piperazine,
1-[2-(2,4-Difluorophenoxy)ethyl]-4-[2-(3-methanesulfonylphenoxy)ethyl]piperazine,
1-[2-(4-Fluorophenoxy)ethyl]-4-[2-(4-trifluoromethanesulfonylphenoxy)ethyl]piperazine,
1-[2-(2-Fluorophenoxy)ethyl]-4-[2-(4-trifluoromethanesulfonylphenoxy)ethyl]piperazine,
1-[2-(2,4-Difluorophenoxy)ethyl]-4-[2-(4-trifluoromethanesulfonylphenoxy)ethyl]piperazine,
1-[2-(4-Fluorophenoxy)ethyl]-4-[2-(3-trifluoromethanesulfonylphenoxy)ethyl]piperazine,
1-[2-(2-Fluorophenoxy)ethyl]-4-[2-(3-trifluoromethanesulfonylphenoxy)ethyl]piperazine,
1-[2-(2,4-Difluorophenoxy)ethyl]-4-[2-(3-trifluoromethanesulfonylphenoxy)ethyl]piperazine,
4-(3-{4-[2-(4-Fluorophenoxy)ethyl]piperazin-1-yl}propoxy)benzonitrile,
4-(3-{4-[2-(2-Fluorophenoxy)ethyl]piperazin-1-yl}propoxy)benzonitrile,
4-(3-{4-[2-(2,4-Difluorophenoxy)ethyl]piperazin-1-yl}propoxy)benzonitrile,
3-(3-{4-[2-(4-Fluorophenoxy)ethyl]piperazin-1-yl}propoxy)benzonitrile,
3-(3-{4-[2-(2-Fluorophenoxy)ethyl]piperazin-1-yl}propoxy)benzonitrile,
3-(3-{4-[2-(2,4-Difluorophenoxy)ethyl]piperazin-1-yl}propoxy)benzonitrile,
1-[2-(4-Fluorophenoxy)ethyl]-4-[3-(4-trifluoromethylphenoxy)propyl]piperazine,
1-[2-(2-Fluorophenoxy)ethyl]-4-[3-(4-trifluoromethylphenoxy)propyl]piperazine,
1-[2-(2,4-Difluorophenoxy)ethyl]-4-[3-(4-trifluoromethylphenoxy)propyl]piperazine,
1-[2-(4-Fluorophenoxy)ethyl]-4-[3-(3-trifluoromethylphenoxy)propyl]piperazine,
1-[2-(2-Fluorophenoxy)ethyl]-4-[3-(3-trifluoromethylphenoxy)propyl]piperazine,
1-[2-(2,4-Difluorophenoxy)ethyl]-4-[3-(3-trifluoromethylphenoxy)propyl]piperazine,
1-[2-(4-Fluorophenoxy)ethyl]-4-[3-(4-methanesulfonylphenoxy)propyl]piperazine,
1-[2-(2-Difluorophenoxy)ethyl]-4-[3-(4-methanesulfonylphenoxy)propyl]piperazine,
1-[2-(2,4-Difluorophenoxy)ethyl]-4-[3-(4-methanesulfonylphenoxy)propyl]piperazine,
1-[2-(4-Fluorophenoxy)ethyl]-4-[3-(3-methanesulfonylphenoxy)propyl]piperazine,
1-[2-(2-Fluorophenoxy)ethyl]-4-[3-(3-methanesulfonylphenoxy)propyl]piperazine,
1-[2-(2,4-Difluorophenoxy)ethyl]-4-[3-(3-trifluoromethanesulfonylphenoxy)propyl]piperazine,
1-[2-(4-Fluorophenoxy)ethyl]-4-[3-(4-trifluoromethanesulfonylphenoxy)propyl]piperazine,
1-[2-(2-Fluorophenoxy)ethyl]-4-[3-(4-trifluoromethanesulfonylphenoxy)propyl]piperazine,
1-[2-(2,4-Difluorophenoxy)ethyl]-4-[3-(4-trifluoromethanesulfonylphenoxy)propyl]piperazine,
1-[2-(4-Fluorophenoxy)ethyl]-4-[3-(3-trifluoromethanesulfonylphenoxy)propyl]piperazine,
1-[2-(2-Fluorophenoxy)ethyl]-4-[3-(3-trifluoromethanesulfonylphenoxy)propyl]piperazine, and
1-[2-(2,4-Difluorophenoxy)ethyl]-4-[3-(3-trifluoromethanesulfonylphenoxy)propyl]piperazine,
or a pharmaceutically acceptable salt of the selected compound, or a stereoisomeric form,
or a polymorphic form of said compound or of said salt.
